(19) United States
(12) Patent Application Publication
(10) Pub. No.: US 2005/0205450 A1
(43) Pub. Date: Sep. 22, 2005

(54) Holder for surgical instruments

(76) Inventor: Robert W. Leitch, Mt. Martha (AU)

Correspondence Address: BARTONY & HARE, LAW & FINANCE BUILDING, SUITE 1801, 429 FOURTH AVENUE, PITTSBURGH, PA, 15219, US

(21) Appl. No.: 10/516,855

(22) PCT Filed: Jun. 5, 2003
(86) PCT No.: PCT/AU03/00705

§ 371 Date: Dec. 3, 2004

(30) Foreign Application Priority Data
Jun. 6, 2002 (AU) PS 2817

(51) Int. Cl. A61B 17/06

(52) U.S. Cl. 206/438; 206/363

(57) ABSTRACT

A holder for surgical instruments, said instruments being of the type having a distal portion for engagement with a patient during surgery and a proximal portion for manipulation by a surgeon, said holder comprising a body (7), a plurality of retainers (5) mounted on or in the body (7) and a separator located between adjacent retainers (5), wherein location of the distal portion of a surgical instrument in or by a retainer (5) substantially separates the distal portion of the surgical instrument against contact with the distal portion of a surgical instrument located in an adjacent retainer (5) and the distal portions of the surgical instrument being substantially enclosed by the retainer (5).

### FIELD OF THE INVENTION

The invention relates to holders for surgical instruments and methods of conducting surgical procedures utilizing the benefits of such holders.

### BACKGROUND OF THE INVENTION

In this specification, where a document, act or item of knowledge is referred to or discussed, this reference or discussion is not an admission that the document, act or item of knowledge or any combination thereof was at the priority date:
(i) part of common general knowledge; or
(ii) known to be relevant to an attempt to solve any problem with which this specification is concerned.

While conducting surgical operations, numerous surgical instruments must be transferred between personnel such as surgeons, assistant surgeons, scrub nurses and scrub technicians. Surgical instruments, by their nature have sharp protuberances, either for cutting or clasping tissue. Such surgical instruments include suture needles, hypodermic syringe needles, scalpels, wires, retractors, forceps and other sharp instruments.

Traditionally to transfer instruments between persons during an operation the instruments are handed directly from one person to another. This involves orienting the instrument in the correct direction so that the person receiving the instrument receives the handle or proximal portion of the instrument rather than the sharp end. However sharps injuries are relatively common using this method since the person handing over the sharp instrument is at risk from the sharp or distal end of the instrument being closest to them or indeed within their hand during transfer.

Another method of transferring instruments between persons during surgery involves placing the instrument in a tray such as a kidney dish or other specifically adapted tray and passing the tray between the persons. This has the disadvantage of wasting time and the person receiving the instrument must still pick it up from the tray without injuring themselves or others. In any event with such formal systems of transfer of instruments between persons involved in surgery, they require at least one person other than the surgeon to effect the transfer.

In addition, it is unusual these days for a surgeon to work with the same team each time he or she is performing an operation. Thus, the team may not be particularly familiar with the way the surgeon works, and which instruments they will require and when and how to pass the instruments to them. This periodically leads to confusion as to which instrument is required or when, and apart from slowing the surgery time down can lead to further sharps injuries.

There are a number of particularly important infectious diseases which can be transferred via sharps injuries. Three of the most important are Human Immunodeficiency irus, Hepatitis B and Hepatitis C. Apart from the obvious deleterious medical effects of serious infectious diseases there are economic and time wasting effects of surgical sharp injuries. If a needle stick or surgical sharp injury occurs during surgery the person affected by the injury will first immediately withdraw from the surgical site, apply first aid to the wound including thorough sterilisation such as scrubbing and soaking with an antiseptic such as povidone iodine. The injured person must then fill in an incident report, and undergo serology to test for their baseline antibody titre for the infectious diseases.

The patient who had been operated on will be counselled after recovering from the anaesthetic and advised that there was a sharps injury during surgery. The patient will be asked whether they are aware whether they carry any infectious disease. Questions will also be asked which are designed to ascertain whether other significant sociological risk factors for these infectious diseases are present. With their consent, and appropriate counselling, serology will also be performed on the surgical patient. What follows is a reasonably lengthy period of follow up with the person injured by the sharp incident. This will involve counselling, time lost from work and further serology to confirm a negative titre for the infectious diseases (after an appropriate time period). The patient may need further serology depending on their sociological history, for example if there is a chance that they were incubating an infectious disease at the time of the injury.

In economic terms such surgical sharp injuries can cost thousands of dollars per incident and have one of the highest economic impacts of all injury types in occupational health and safety reports of hospitals and allied medical institutions.

In addition, the traditional methods and traditional surgical trays do not lend themselves to efficient and safe surgery performed by a single surgeon without assistance. In the past, surgeons wishing to conduct surgery in this manner would use instruments on the tray, laid out by the surgeon or a nurse. However, during surgery, such instruments can tend to become jumbled together, particularly in situations where rapid manoeuvres, urgent haemostasis or retraction etc., is required and instruments are quickly dropped, or picked up from the surgical tray.

It is an object of the present invention to provide a holder for surgical instruments which decreases the chances of sharps injuries compared with traditional instrument handling methods described above.

It is a still further object of the present invention to provide a holder for surgical instruments which simplifies the surgical process for an assisted surgeon or a single surgeon operating without assistance.

### SUMMARY OF THE INVENTION

Accordingly, the present invention provides a holder for surgical instruments, said instruments being of the type having a distal portion for engagement with a patient during surgery and a proximal portion for manipulation by a surgeon, said holder comprising a body, a plurality of retainers mounted on or in the body and a separator located between adjacent retainers, wherein location of the distal portion of a surgical instrument in or by a retainer substantially separates the distal portion of the surgical instrument against contact with the distal portion of a surgical instrument located in an adjacent retainer and the distal portions of the surgical instrument being substantially enclosed by the retainer.

A holder according to the present invention can be used with almost all known surgical instruments. It is merely necessary to alter the size of the retainer to suit the surgical instrument of interest. For example, the invention may be used to hold scalpels or scalpel blades, forceps, scissors, clamps, haemostats, needle drivers etc.

The distal portion of the surgical instruments is correspondingly the blade of a scalpel, the grasping portion of forceps, clamps or haemostats, the blades of a pair of scissors and the grasping portion (and potentially the blades) of needle drivers.

The surgeon may directly or indirectly manipulate the proximal portion of the surgical instrument. An example of direct manipulation is where the surgeon grasps the proximal portion of the surgical instrument with his or her hand. Indirect manipulation includes any application where the instrument is not directly contacted by the surgeon, for example, a surgeon picking up a scalpel blade placed in a retainer by means of a scalpel handle will be indirectly manipulating the scalpel blade. Thus where the instrument is engaged by another instrument or piece of equipment, then the portion so engaged is the proximal portion. For example, the non-blade end of a scalpel blade, or the hub end of a needle.

A retainer according to this invention may take any suitable form. For example, it may retain by grasping the instruments, by hanging or hooking the instrument on the retainer, or it may be a recess or well into which the instrument (or a portion of it) is placed. A separator according to this embodiment may take any suitable form. For example, it may be a component adapted to keep adjacent instruments distracted from one another, equally it may simply be a wall or other portion of the body of the holder such as a part of a retainer.

However, location of the distal portion of a surgical instrument in or by a retainer must substantially separate the distal portion of the surgical instrument to minimise the risk of direct contact with other surgical instruments located in adjacent retainers.

As indicated above, the distal portions of the surgical instruments are substantially enclosed by the retainers. This further minimises the risk of sharps injury to a surgeon, assistant surgeon, or other assistants (etc) who might otherwise accidentally touch the distal portion in its retainer. By ‘substantially enclosed’, it is meant that the instruments are sufficiently enclosed so as to effectively separate the distal portions of surgical instruments. However, it is contemplated that this does not preclude an embodiment in which the distal end of the retainer comprises an aperture as described below (for example, to allow egress or drainage of fluid from the retainer).

According to a still further preferred embodiment, retainers are configured such that when retained, instruments are oriented such that their proximal portion projects from the body of the holder. This enables the proximal portion to be more readily accessed by the surgeon or assistant and thus reduces wastage of time.

According to another preferred embodiment, the retainer defines a cavity in the body of the holder. A retainer according to this embodiment may or may not be integral with the body of the holder, it may also be separately formed and yet still define a cavity in the body of the holder. Where the retainer is separately formed, then the retainer can be engaged with and fastened to the body of the holder. For example, there may be provided an engagement means to allow engagement of the retainer and the holder and a fastener to fasten the retainer and holder. Any suitable engagement means and any suitable fastener can be used. According to one embodiment, the retainer and holder may slidingly engage each other and may be fastened by interference fit. According to another embodiment, the body of the retainer is passed through an aperture in the holder and fastened to the body of the holder. Fastening of such a retainer to the body of the holder may be by any suitable means. According to one embodiment, the retainer is fastened to the body of the holder by means of an adhesive strip.

Retainers according to this preferred embodiment may be integral with or separate from other retainers. According to a most preferred embodiment, the retainers are separate and can be separately reversibly fastened in apertures in the body of the holder. Such retainers may be removed after surgery, sterilised and replaced or substituted with new retainers.

Where the retainer is oriented appropriately, the distal portion of an instrument may be kept in the retainer by gravity. Such an embodiment allows instruments to be quickly placed in and removed from the retainer. A cavity according to this embodiment may be enclosed by or may define an aperture. Where there is an aperture, the instrument may optionally extend through the aperture. Such an embodiment has the advantage of assisting with drainage of waste material through the aperture and away from contact with the instrument.

According to a most preferred embodiment, the retainers are cavities and the cavities are elongated and tapered. Most preferably, the cavities are elongate tapering with an oval or bi-lobed cross-section which is preferably elongate. However, a square, rectangular or other cross-sectional shape to accommodate a specific instrument will also be suitable. The cavities may be of any suitable dimension. According to one embodiment, the cavities are approximately 4 cm in depth and the bi-lobed cross section tapers from approximately 3 cm to approximately 2 cm. Most preferably, the cavities are formed separately from the body of the holder.

It has been found that cavities with this shape are able to retain a broad range of surgical instruments of the type comprising matching paired distal components, for example the two arms of a pair of forceps, or the two blades of a pair of scissors. The respective arms or blades thus slide into different lobes of the cavity as the instrument is placed in the cavity.

Preferably the wall of each cavity (retainer) defines an aperture at its tapered end so as to enable fluid on a surgical instrument placed in it to fall through the aperture rather than pooling in the bottom of the cavity. Such pooling of fluids (for example blood) increases the chances of bacterial growth and thus contamination of wounds.

Certain embodiments of the present invention may not require such an aperture. An example of such an embodiment is where the length of the elongate cavity is substantially longer than the length of the distal portion of the surgical instrument. Similarly, if the surgical instrument is to be used with sterile fluids, then it is not as necessary to ensure drainage from them. There will be situations where it is required that the distal portion of the surgical instrument must be kept in a fluid (such as a sterile fluid) which will necessitate that there is no aperture in the tapered end of the cavity.

The retainer may also retain the instrument by engaging it. This may be any suitable form of engagement. The retainer may grasp the instrument, or it may cause sufficiently increased friction (for example, an interference fit) so as to maintain the distal portion of the instrument in the retainer.

A holder according to one embodiment of the present invention may sit directly on the surgical tray or surgical drapes or it may sit on a base. A holder without a base has advantages such as being easier to manufacture and easier to clean if it is to be re-used. Where the holder sits on the base, it may either be integral with the base or it may be separate from the base. Preferably the holder and base comprise complementary portions to enable engagement.

According to another preferred embodiment, the holder comprises a holding portion and a collecting portion. The body of the holding portion of this embodiment further comprises a plurality of retainers mounted on or in the body and a separator located between adjacent retainers wherein location of a distal portion of a surgical instrument in or by a retainer separates the distal portion of the surgical instrument against contact with other surgical instruments located in adjacent retainers.

The collecting portion of this embodiment may take any suitable form. For example, it may comprise a vessel for collecting fluids, or it may comprise a base as described above. Such a collecting portion may be integral with the holding portion, or it may not. According to one preferred embodiment, the collecting portion is adapted to collect fluids from instruments in the retainers.

The collecting portion may also be used to collect miscellaneous surgical waste, for example, body fluids, excised tissue, used swabs etc. Where the embodiment includes such a collecting portion then it is preferable for the holding portion to define at least one aperture suitable for passing the surgical waste through. A preferable form of this embodiment comprises a detachable collecting portion to enable it to be separately disposed of, or alternatively emptied using aseptic technique and returned to the surgical tray.

An aperture according to this embodiment is most preferably adapted to enable a variety of waste material to pass through it. In addition, such an aperture is preferably adapted to enable the passage of surgical or medical sharps. According to a particularly preferred embodiment, such an aperture is adapted to enable removal of surgical sharps.

In one particularly preferred embodiment, the body of the holding portion defines an aperture wherein at least one portion of the body projects into the aperture and said portion comprises a lip extending substantially perpendicular to the plane of the body defining the aperture so as to engage the medical sharp for removal from the medical or surgical implement.

According to a particularly preferred embodiment comprising the collecting portion, the instruments are held in the retainers so as to not contact the collecting portion. Preferably the retainers suspend the instruments above the collecting portion and preferably the retainers comprise indentations in the body of the holding portion (or holder). Such cavities or indentations preferably define an aperture in order to allow fluid or other material on the instruments to pass into the collecting portion. Preferably such material simply falls from the instruments into the collecting portion.

According to another preferred embodiment, the collecting portion comprises a tray. The holding portion of a holder according to this embodiment may engage such a tray in any suitable way. For example, the holding portion may simply sit over the tray, or the tray may be slidably engaged with the holding portion in a similar manner to a drawer in a chest of drawers.

Where the collecting portion is to be used to collect fluids, then preferably it comprises a fluid absorbent surface, such as a fluid absorbent mat.

According to another preferred embodiment, the holding portion further comprises a portion for holding other surgical items. Such items may include for example, surgical needles, suture material, implants, etc. Preferably such a portion comprises an indentation in the body of the holder or holding portion. Such a portion may comprise an adhesive or magnetic strip to temporarily hold to surgical items. This enables needles and blades to be laid out and then picked up as necessary by the surgeon and simplifies the needle count at the end of surgery.

A holder according to the present invention may be designed for single use and subsequent disposal or for multiple use after re-sterilisation. To this end, the holder may be manufactured from any suitable material. For example, it may be manufactured from stainless steel, poly-vinyl chloride polymer, polyethylene, polypropylene, composite polymer materials, other plastic materials or any other material suitable for multiple use after re-sterilisation.

Holders according to the present invention may be adapted for particular applications, for example, holders with particular features may be adapted for orthopaedics, ophthalmology, soft tissue surgery, microsurgery, veterinary and all surgical disciplines as well as dental work. In a preferred embodiment, holders may be adapted for particular surgical procedures, for example, a particular surgical procedure may require the use of particular surgical instruments and in a particular order.

In one embodiment which is particularly useful for microsurgical procedures, the retainers of the holder are blind ending cavities in that there is no aperture defined in the end of the cavity. Such an embodiment allows the cavity of the retainer to be filled with fluid to retain microsurgical instruments. Preferably the retainer comprises a pad for the microsurgical instruments to sit on. Such a pad is most preferably soft so as to minimise damage to the sharp portions of the microsurgical instruments. The fluid filling the cavity may be of any suitable type. The fluid may be any fluid suitable for intravenous administration, for example, it may be lactated ringers solution or isotonic (0.9%) saline.

Preferably the holder is shaped so that the holder or the holding portion of the holder is stackable with a plurality of like-shaped holders (or holding portions). Similarly, preferably the collecting portion is also stackable with other like-shaped containing portions. The holding portion and/or collecting portion may also be stackable with other items. The ability to stack in this manner greatly assists with storage and transport of such holders.

According to a particularly preferred embodiment, the holder comprises an engagement portion to engage another surgical tray or holder. Preferably the engagement portion enables complementary engagement of the engagement portions of holders according to the present invention. Such engagement enables holders to be put together in combination for particular applications. For example, it may be necessary to use holders with particular retainers for a particular surgical procedure. The holders can be arranged in any suitable fashion so as to maximise the ease of use for the surgeon.

The holders may be engaged in any suitable way, for example, they may be stacked, or engaged next to each other or in tiers. The means of engagement between the holders can be of any suitable type. For example, they may slidingly engage, they may snap into engagement or simply fit together. According to one embodiment, a holder according to the present invention is adapted to engage a standard surgical tray.

According to a further preferred embodiment, there is provided a holder for surgical instruments comprising a body adapted so that the holder or the holding portion of the holder is stackable with a plurality of like-shaped holders (or holding portions). Similarly, preferably the collecting portion is also stackable with other like-shaped containing portions. The holding portion and/or collecting portion may also be stackable with other items. The ability to stack in this manner greatly assists with storage and transport of such holders.

According to a second aspect of the invention there is provided a device for removal of medical sharps from medical implements comprising a body defining an aperture wherein at least one portion of the body projects into the aperture and said portion comprises a lip extending substantially perpendicular to the plane of the body defining the aperture so as to engage the medical sharp for removal from the medical implement. Preferably the aperture is a generally rounded rectangular shape and preferably the rounded rectangle is missing one corner due to the projection of the body into the aperture. Preferably the lip extends less than half way around the perimeter of the aperture and preferably it extends half way across the projection from the body into the aperture. According to the most preferred embodiment of this aspect of the invention, the lip extends perpendicularly to the plane of the body from which the aperture is defined.

According to another embodiment, the projection of the body comprises a tapered or serrated edge to enable removal of screw-threaded sharps from medical instruments. This embodiment is particularly useful for removing screw-on sharps and components such as screw-on needles and catheters. Alternatively, another portion of the aperture may comprise a serrated edge to enable removal of screw-threaded sharps from medical instruments.

According to a third aspect of the present invention, there is provided a retainer for a holder for surgical instruments according to the present invention. A retainer according to this aspect of the invention may take any suitable form. For example, it may retain by grasping the instruments, by hanging or hooking the instrument on the retainer, or it may be a recess or well into which the instrument (or a portion of it) is placed. A separator according to this embodiment may take any suitable form. For example, it may be a component adapted to keep adjacent instruments distracted from one another, equally it may simply be a wall such as a part of a retainer, or other portion of the body of the holder.

However, location of the distal portion of a surgical instrument in or by a retainer must substantially separate the distal portion of the surgical instrument to minimise the risk of direct contact with other surgical instruments located in adjacent retainers.

According to one preferred embodiment, the distal portions of the surgical instruments are substantially enclosed by the retainers. This further minimises the risk of sharps injury to a surgeon or assistant surgeon (etc) who might otherwise accidentally touch the distal portion in its retainer. By ‘substantially enclosed’, it is meant that the instruments are sufficiently enclosed so as to effectively separate the distal portions of surgical instruments. However, it is contemplated that this does not preclude an embodiment in which the distal end of the retainer comprises an aperture as described below (for example, to allow egress or drainage of fluid from the retainer).

According to a still further preferred embodiment, retainers are configured such that when retained, instruments are oriented such that their proximal portion projects from the body of the holder. This enables the proximal portion to be more readily accessed by the surgeon or assistant and thus reduces wastage of time.

According to another preferred embodiment, the retainer defines a cavity in the body of the holder. A retainer according to this embodiment may be integral with the body of the holder, or it may be separate. Where the retainer is separate, then preferably the retainer can be readily engaged with and fastened to the holder. For example, there may be provided an engagement means to allow engagement of the retainer and the holder and a fastener to fasten the retainer and holder. Any suitable engagement means and fastener can be used. However, according to one preferred embodiment, the retainer and holder may be slidingly engaged and may be fastened by interference fit.

Retainers according to this preferred embodiment may be integral with or separate from other retainers. According to a most preferred embodiment, the retainers are separate and can be separately reversibly fastened in apertures in the body of the holder. Such retainers may be removed after surgery, sterilised and replaced or substituted for new retainers.

Where the retainer is oriented appropriately, the distal portion of the instrument may be kept in the retainer by gravity. Such an embodiment allows instruments to be quickly placed in and removed from the retainer. The cavity according to this embodiment may be enclosed by or may define an aperture. Where there is an aperture, the instrument may optionally extend through the aperture. Such an embodiment has the advantage of assisting with drainage of waste material through the aperture and away from contact with the instrument.

According to a most preferred embodiment, the retainers are cavities and the cavities are elongated and tapered. Most preferably, the cavities are elongate tapering with an oval or bi-lobed cross-section which is preferably elongate. However, a square, rectangular or any other cross-sectional shape to accommodate a specific instrument will also be suitable.

The cavities may be of any suitable dimension. According to one embodiment, the cavities are approximately 4 cm in depth and the bi-lobed cross section tapers from approximately 3 cm to approximately 2 cm.

It has been found that cavities with this shape are able to retain a broad range of surgical instruments of the type comprising matching paired distal components, for example the two arms of a pair of forceps, or the two blades of a pair of scissors. The respective arms or blades thus slide into different lobes of the cavity as the instrument is placed in the cavity.

Preferably the wall of each cavity (retainer) defines an aperture at its tapered end so as to enable fluid on a surgical instrument placed in it to fall through the aperture rather than pooling in the bottom of the cavity. Such pooling of fluids (for example blood) increases the chances of bacterial growth and thus contamination of wounds.

Certain embodiments of the present invention may not require such an aperture. An example of such an embodiment is where the length of the elongate cavity is substantially longer than the length of the distal portion of the surgical instrument. Similarly, if the surgical instrument is to be used with sterile fluids, then it is not as necessary to ensure drainage from them. There will be situations where it is required that the distal portion of the surgical instrument must be kept in a fluid (such as a sterile fluid) which will necessitate that there is no aperture in the tapered end of the cavity.

The retainer may also retain the instrument by engaging it. This may be any suitable form of engagement. The retainer may grasp the instrument, or it may cause sufficiently increased friction (for example, an interference fit) so as to maintain the distal portion of the instrument in the retainer.

According to a fourth aspect of the invention there is provided a method of instrument use during surgery comprising the steps of
1. utilizing a surgical instrument during surgery;
2. locating the surgical instrument in a retainer of a surgical instrument holder as described above;
3. removing the surgical instrument from the holder for further surgical use; and
4. replacing the surgical instrument in the holder following suitable surgical use.

In one preferred embodiment, the nurse or surgical assistant is responsible for managing the process of use of the surgical instrument holder. This includes ensuring that the holder is placed in the correct position and the correct instruments are located in the correct retainers prior to and during surgery, with instruments interchanged as needed and cleaned as required.

According to a particularly preferred embodiment, the surgical holder according to this aspect of the invention is as described above.

This aspect of the invention enables surgery to be conducted in a more efficient manner. In particular, by having a holder to retain instruments during surgery, it enables the surgeon to pick up and replace instruments as required without the need to ask for them or for transfer of instruments between persons which increases the risk of sharps injuries occurring.

According to a particularly preferred embodiment, the holder utilised in the method of instrument use during surgery according to the present invention is adapted for use with instruments being of the type having a distal portion for engagement with a patient during surgery and a proximal portion for manipulation by a surgeon, and the holder comprises a body, a plurality of retainers mounted on or in the body and a separator located between adjacent retainers, wherein location of the distal portion of a surgical instrument in or by a retainer substantially separates the distal portion of the surgical instrument against contact with the distal portion of a surgical instrument located in an adjacent retainer.

The method according to the present invention can be used with almost all known surgical instruments. For example, the invention may be used for surgical procedures involving scalpels or scalpel blades, forceps, scissors, clamps, haemostats, needle drivers etc.

The surgeon may directly or indirectly manipulate the proximal portion of the surgical instrument. An example of direct manipulation is where the surgeon grasps the proximal portion of the surgical instrument with his or her hand. Indirect manipulation includes any application where the instrument is not directly contacted by the surgeon, for example, a surgeon picking up a scalpel blade placed in a retainer by means of a scalpel handle will be indirectly manipulating the scalpel blade. Thus where the instrument is engaged by another instrument or piece of equipment, then the portion so engaged is the proximal portion. For example, the non-blade end of a scalpel blade, or the hub end of a needle.

A retainer used according to the method of this invention may take any suitable form. For example, it may retain by grasping the instruments, by hanging or hooking the instrument on the retainer, or it may be a recess or well into which the instrument (or a portion of it) is placed. A separator according to this embodiment may take any suitable form. For example, it may be a component adapted to keep adjacent instruments distracted from one another, equally it may simply be a wall or other portion of the body of the holder such as a part of a retainer.

However, location of the distal portion of a surgical instrument in or by a retainer must substantially separate the distal portion of the surgical instrument to minimise the risk of direct contact with other surgical instruments located in adjacent retainers.

According to one preferred embodiment, the distal portions of the surgical instruments are substantially enclosed by the retainers when they are located in them. This further minimises the risk of sharps injury to a surgeon or assistant surgeon (etc) who might otherwise accidentally touch the distal portion in its retainer. By ‘substantially enclosed’, it is meant that the instruments are sufficiently enclosed so as to effectively separate the distal portions of surgical instruments. However, it is contemplated that this does not preclude an embodiment in which the distal end of the retainer comprises an aperture as described below (for example, to allow egress or drainage of fluid from the retainer).

According to a still further preferred embodiment, retainers are configured such that retention of the instruments orients them such that their proximal portion projects from the body of the holder. This enables the proximal portion to be more readily accessed by the surgeon or assistant and thus reduces wastage of time.

According to another preferred embodiment, the retainer into which the instrument is located defines a cavity in the body of the holder. A retainer according to this embodiment may be integral with the body of the holder, or it may be separate. Where the retainer is separate, then preferably the retainer can be readily engaged with and fastened to the holder. For example, there may be provided an engagement means to allow engagement of the retainer and the holder and a fastener to fasten the retainer and holder.

Where the retainer is oriented appropriately, the distal portion may be kept in the retainer by gravity. Such an embodiment allows instruments to be quickly placed in and removed from the retainer. The cavity according to this embodiment may be enclosed by or may define an aperture. Where there is an aperture, the instrument may optionally extend through the aperture. Such an embodiment has the advantage of assisting with drainage of waste material through the aperture and away from contact with the instrument.

The instrument may also be retained in the retainer by engagement with it. This may be any suitable form of engagement. The retainer may grasp the instrument, or it may cause sufficiently increased friction (for example, an interference fit) so as to maintain the distal portion of the instrument in the retainer.

According to another preferred embodiment, there is a further step of collecting materials in a collecting portion associated with the holder. Preferably the holder comprises the collecting portion. The collecting portion of this embodiment may take any suitable form. For example, it may comprise a vessel for collecting fluids, or it may comprise a base as described above. Such a collecting portion may be integral with the holding portion, or it may not. According to one preferred embodiment, the collecting portion is adapted to collect fluids from instruments in the retainers.

The collecting portion may also be used to collect miscellaneous surgical waste, for example, body fluids, excised tissue, used swabs etc. Where the embodiment includes such a collecting portion then it is preferable for the holding portion to define at least one aperture suitable for passing the surgical waste through. A preferable form of this embodiment comprises a detachable collecting portion to enable it to be separately disposed of, or alternatively emptied using aseptic technique and returned to the surgical tray.

An aperture according to this embodiment is most preferably adapted to enable a variety of waste material to pass through it. In addition, such an aperture is preferably adapted to enable the passage of surgical or medical sharps. According to a particularly preferred embodiment, such an aperture is adapted to enable removal of surgical sharps.

In one particularly preferred embodiment, there is provided the further step of removing a surgical or medical sharp comprising engaging the sharp with a sharp removal means comprising a body defining an aperture wherein at least one portion of the body projects into the aperture and said portion comprises a lip extending substantially perpendicular to the plane of the body defining the aperture so as to engage the medical sharp for removal from the medical or surgical implement.

According to a particularly preferred embodiment comprising the collecting step, the instruments are held in the retainers so as to not contact the collecting portion. Preferably the retainers suspend the instruments above the collecting portion and preferably the retainers comprise indentations in the body of the holding portion (or holder). Such cavities or indentations preferably define an aperture in order to allow fluid or other material on the instruments to pass into the collecting portion. Preferably such material simply falls from the instruments into the collecting portion.

Where the collecting portion is to be used to collect fluids, then preferably it comprises a fluid absorbent surface, such as a fluid absorbent mat.

According to another preferred embodiment, there is provided the further step of retaining other surgical items in a portion of the holding portion. Such other surgical items may include for example, surgical needles, suture material, implants, etc. Preferably such a portion comprises an indentation in the body of the holder or holding portion. Such a portion may comprise an adhesive or magnetic strip to temporarily hold to surgical items. This enables needles and blades to be laid out and then picked up as necessary by surgeon and simplifies with the needle count at the end of surgery.

According to the present method, a holder may be designed for single use and subsequent disposal or for multiple use after re-sterilisation. To this end, the holder may be manufactured from any suitable material. For example, it may be manufactured from stainless steel, polyvinyl chloride polymer, polyethylene, polypropylene, composite polymer materials or other plastic materials.

The method according to the present invention is suitable for many different surgical applications. For example, the method of use of such holders may be adapted for orthopaedics, ophthalmology, soft tissue surgery, microsurgery, veterinary and all surgical disciplines as well as dental work. In a preferred embodiment, the method of use of a holder may be adapted for a particular surgical procedure, for example, a particular surgical procedure may require the use of particular surgical instruments and in a particular order.

In one embodiment which is particularly useful for microsurgical procedures, the instruments are retained in retainers which are blind ending cavities in that there is no aperture defined in the end of the cavity. Such an embodiment allows the cavity of the retainer to be filled with fluid to retain microsurgical instruments. Preferably the retainer comprises a pad for the microsurgical instruments to sit on. Such a pad is most preferably soft so as to minimise damage to the sharp portions of the microsurgical instruments. The fluid filling the cavity may be of any suitable type. The fluid may be any fluid suitable for intravenous administration, for example, it may be lactated ringers solution or isotonic (0.9%) saline.

According to a particularly preferred embodiment, the method comprises an initial step of assembling the holder from various components. Holders for use with this method will comprise an engagement portion to engage another surgical tray or holder. Preferably the engagement portion enables complementary engagement of the engagement portions of holders according to the present invention. Such engagement enables holders to be put together in combination for particular applications. For example, it may be necessary to have holders with particular retainers for a particular surgical procedure. The holders can be arranged in any suitable fashion so as to maximise the ease of use for the surgeon.

The holders may be engaged in any suitable way, for example, they may be stacked, or engaged next to each other or in tiers. The means of engagement between the holders can be of any suitable type. For example, they may slidingly engage, they may snap into engagement or simply fit together. According to one embodiment, a holder according to the present invention is adapted to engage a standard surgical tray.

### DESCRIPTION OF THE DRAWINGS

The invention will now be further explained and illustrated by reference to the accompanying drawings in which:

FIG. 1 is a perspective view of a holder according to one form of the invention;

FIG. 2 is a perspective view of a collecting portion according to one form of the invention;

FIG. 3 is a perspective view of the underside of the holding portion of the holder of FIG. 1;

FIG. 4 is a plan view of a holder according to another form of the invention;

FIG. 5 is a perspective view of the holder of FIG. 4;

FIG. 6 is a side/perspective view of the holder of FIG. 4;

FIG. 7 is a side view of the holder of FIG. 4;

FIG. 8 is a plan view of the holder of FIG. 4, but without the retainer inserts;

FIG. 9 is a perspective view of the holder of FIG. 8;

FIG. 10 is a side/perspective view of the holder of FIG. 8;

FIG. 11 is a side view of the holder of FIG. 8;

FIG. 12 is a plan view of a retainer according to one aspect of the invention;

FIG. 13 is a perspective view of three different sized retainers as depicted in FIG. 12;

FIG. 14 is a side view of the retainer of FIG. 12;

FIG. 15 is a longitudinal side view of the retainer of FIG. 12;

FIG. 16 is a plan view of a preferred form of a surgical sharp remover according to one aspect of the invention; and

FIG. 17 is a plan view of the cut-out section of the holder which is removed when the aperture of FIG. 16 is cut into the body of a holder according to the present invention.

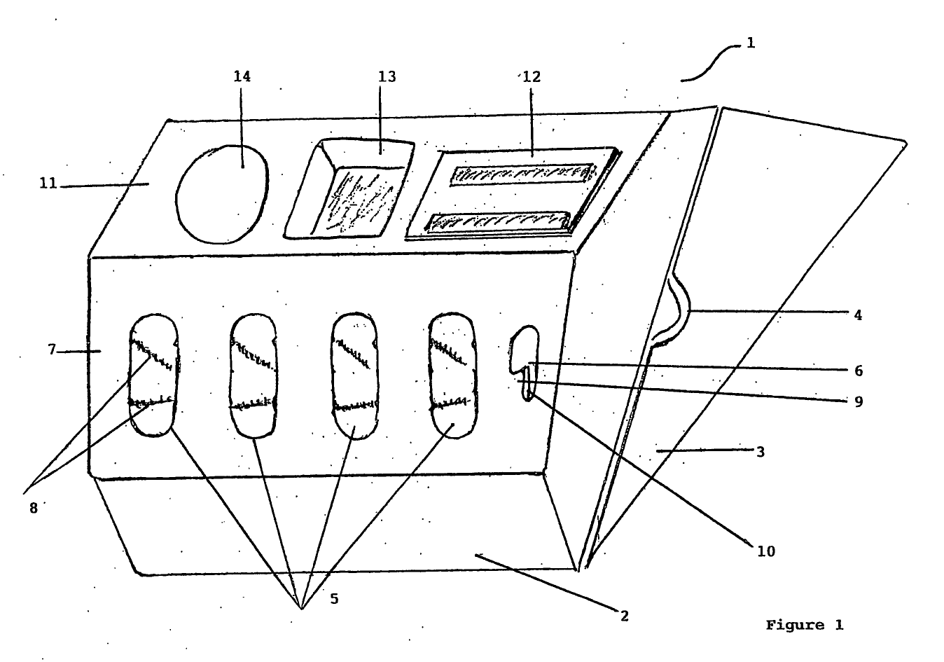

FIGS. 1, 5 and 9 are perspective views of holders shown generally at 1. The holder of FIG. 1 comprises a holding portion and collecting portion according to the present invention. Holding portion 2 and collecting portion 3 (in FIG. 1 only) which are fitted snugly together. Indentation 4 in the wall of collecting portion 3 serves to enable someone using the invention to readily lift holding portion 2 away from collecting portion (or base) 3. It is apparent that the collecting portion 3 is separate from the holding portion 2 and each comprise complementary portions to enable engagement. Furthermore, it will be appreciated that a holder according to the present invention may comprise a holding portion (such as that depicted at 2) without a corresponding collecting portion (such as that depicted at 3).

The holders depicted do not include an engagement portion to engage another surgical holder or tray, but as discussed above, other preferred embodiments include such portions. Such engagement enables holders to be put together in combination for particular applications. For example, it may be necessary to have holders with particular retainers for a particular surgical procedure. The holders can be arranged in any suitable fashion so as to maximise the ease of use for the surgeon.

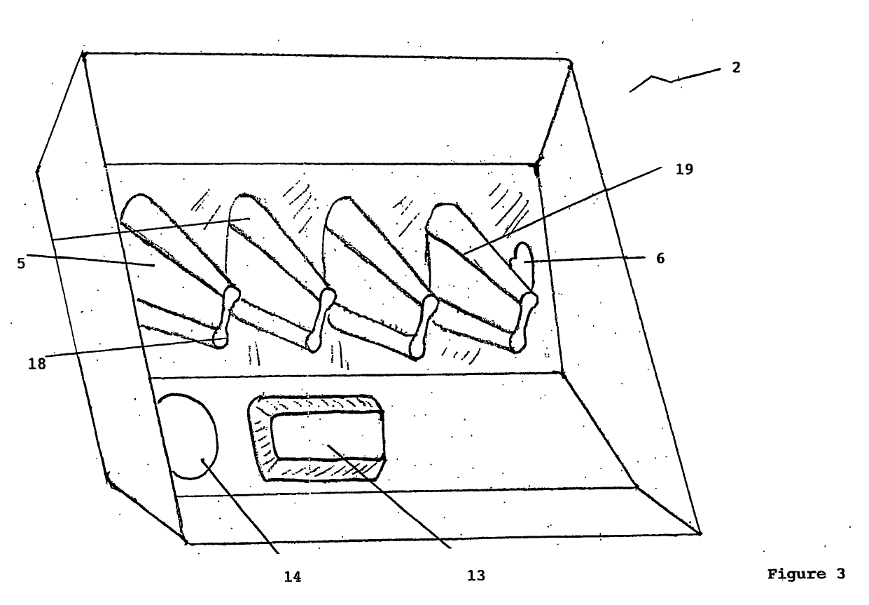

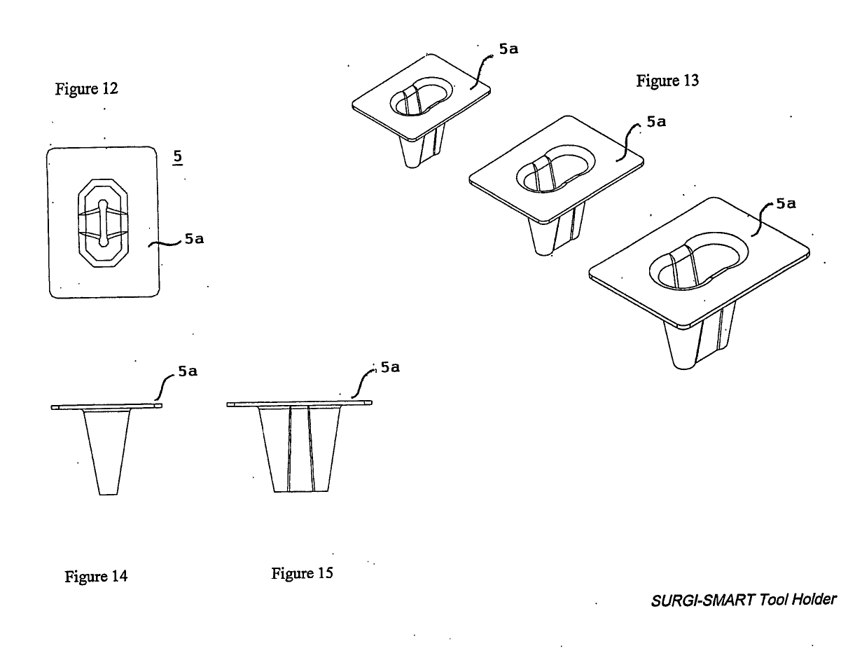

Holding portion 2 in FIGS. 1, 5 and 9 comprises retainers 5 and aperture 6. Retainers 5 comprise cavities in the body 7 of the holding portion 2 of holder 1. The retainers 5 of FIG. 1 are integral with the body of holding portion 2, whereas the retainers 5 of FIG. 5 are removable and are depicted in FIGS. 12, 13, 14 and 15. FIG. 9 shows the holder of FIG. 5 without retainers 5.

Of course, a retainer according to the present invention may take any suitable form. For example, it may retain by grasping the instruments, by hanging or hooking the instrument on the retainer, or it may be a recess or well into which the instrument (or a portion of it) is placed. Similarly, a separator according to this embodiment comprises a portion of the body of the holder. However, it may take any suitable form such as a component adapted to keep adjacent instruments distracted from one another, equally it may simply be a wall.

The cavities in retainers 5 of holder 1 of the present invention are configured such that where the retainer is oriented appropriately, the distal portion of a surgical instrument may be kept in a cavity by gravity. Such an embodiment allows instruments to be quickly placed in and removed from the retainer. Other methods of retaining the instruments are also possible according to the present invention. For example, the retainer may also retain the instrument by engaging it. This may be any suitable form of engagement. The retainer may grasp the instrument, or it may cause an interference fit so as to maintain the distal portion of the instrument in the retainer.

The present embodiment comprises apertures 18 in cavities 5. In the present embodiment, instruments do not necessarily extend through the aperture, though this is possible (for example with elongate scalpel blades). Such an embodiment has the advantage of assisting with drainage of waste material through the aperture and away from contact with the instrument and protecting the tips of delicate and sharp instruments from damage. For example, according to this embodiment, a scalpel blade will be retained in a fashion and angle so as to not blunten the blade margin. A pair of fine forceps or scissor tips will not be impinged upon or damaged.

Certain embodiments of the present invention may not require such an aperture.

It is apparent that in use, distal portions of the surgical instruments will be substantially enclosed by the retainers 5 and configured such that when retained, instruments are oriented such that their proximal portion projects from the body of the holder. This enables the proximal portion to be more readily accessed by the surgeon or assistant and thus reduces wastage of time. By ‘substantially enclosed’, it is meant that the instruments are sufficiently enclosed so as to effectively separate the distal portions of surgical instruments. However, it is contemplated that this does not preclude an embodiment in which the distal end of the retainer comprises an aperture as described below (for example, to allow egress or drainage of fluid from the retainer).

As can be seen by broken lines 8 in FIG. 1 and as depicted in FIGS. 12 to 15, retainers 5 are elongate tapered in shape with a bi-lobed cross section so as to assist in retaining the distal portion of surgical instruments. Such retainers can be used with almost all known surgical instruments. It is merely necessary to alter the size of the cavity in the retainer to suit the surgical instrument of interest. For example, this embodiment may be used to hold scalpels or scalpel blades, forceps, scissors, clamps, haemostats, needle drivers etc., as well as specialised instruments such as chisels, mallets, drills, fibreoptic scopes, motor driven equipment etc. Retainers 5 are able to retain a broad range of surgical instruments of the type comprising matching paired distal components, for example the two arms of a pair of forceps, or the two blades of a pair of scissors. The respective arms or blades thus slide into different lobes of the retainers 5 as the instrument is placed in the cavity.

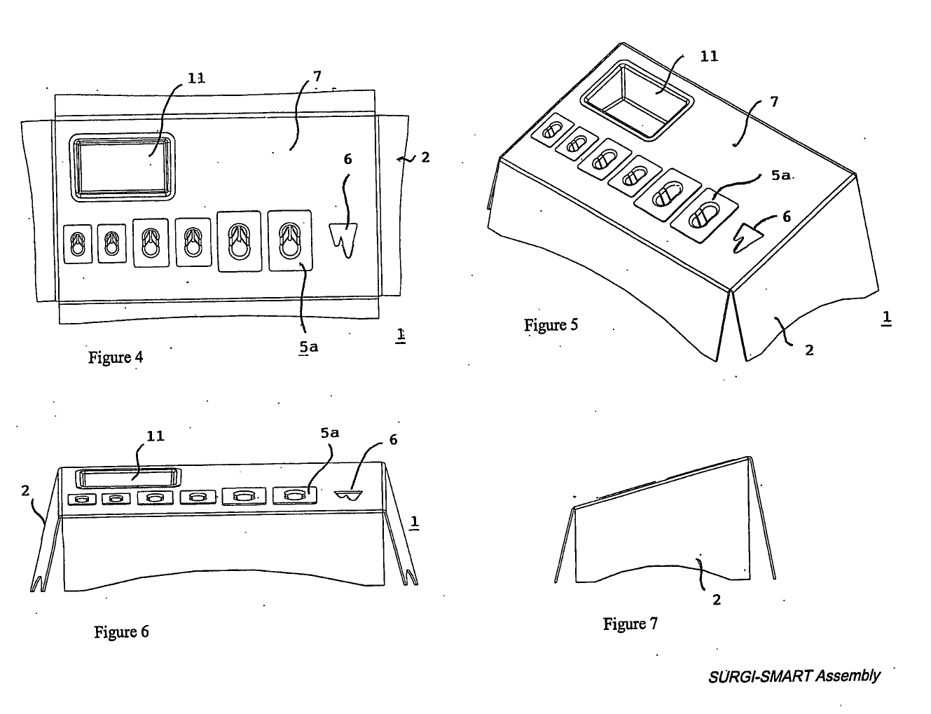

The dimensions of the retainers of FIG. 1 are approximately 4 cm in depth and the bi-lobed cross section tapers from approximately 3 cm to approximately 2 cm. However, as depicted in FIGS. 4, 5 and 13, retainers 5 may come in a variety of sizes. Flange 5a on retainers 5 enables them to engage with the holder irrespective of the size of the cavity in the retainer. FIGS. 8 and 9 depict a holder with apertures 5b through which the cavity portion 5c of retainers 5 are placed. Flange 5a on retainers 5 engages with the portion of the holder surrounding aperture 5b in the holder. Any suitable fastening means may be used to fasten retainers 5 to the holder. For example, an adhesive strip may be attached to the underside of flanges 5a so that retainers 5 can be adhered to the body of the holder.

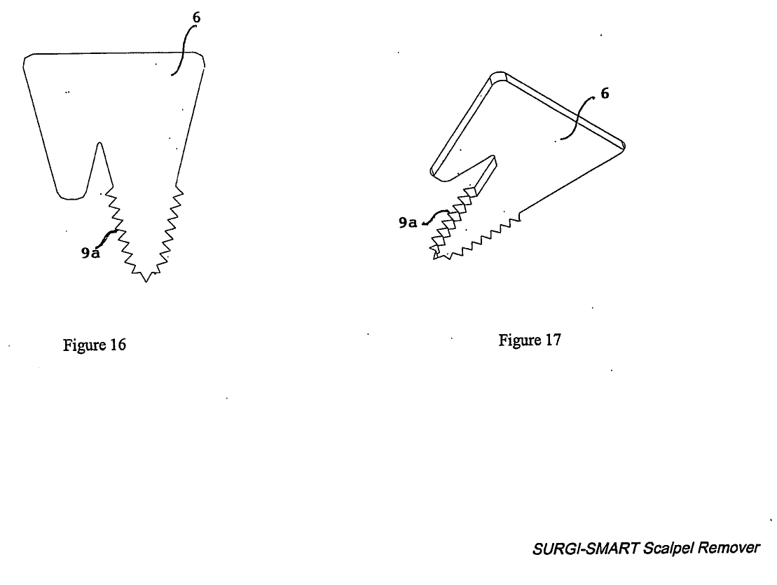

In FIGS. 1 and 16, aperture 6 is defined in body 7 and portion 9 of body 7 projects into aperture 6. Portion 9 comprises lip 10 which extends substantially perpendicular to the plane of body 7 so as to enable engagement of surgical or medical sharps for removal from the surgical instrument or medical implement. The embodiment of aperture 6 depicted in FIGS. 16 and 17 demonstrates a serrated edge 9a cut into aperture 6 which enables screw-on sharps to be grasped and removed in a relatively simple manner and with one hand.

In FIGS. 1, 4 and 5, body 7 further comprises portion 11 for holding other surgical items. As can be seen by a comparison between FIGS. 4 and 5 as against FIGS. 8 and 9, portion 11 may be an insert through an aperture 11a in body 7 in the holder. According to this embodiment, portion 11 may be as simple as a small tray with a lip which sits over aperture 11a.

As depicted in FIG. 1, portion 11 may comprise a pad 12 which is an adhesive strip to adhere to surgical sharps or needles. However, the function of this strip may equally be performed by a magnetic strip or by another suitable means or a combination of more than one means. Indentation 13 in body 7 enables small surgical items to be placed on the holder and it may comprise foam, magnetic adhesive or suitable combinations. This might include scalpel blades or small swabs or any other relevant items. For example, surgical clips or implants.

In embodiments in which there is a collecting portion, such as in FIG. 1, aperture 14 defined in body 7 enables used swabs and other surgical waste such as excised tissue to be dropped through holding portion 2 into collecting portion 3. The aperture according to this embodiment is adapted to enable a variety of waste material including surgical or medical sharps to pass through it. There may be more than one such aperture and there may be variations in the configuration of such apertures will fall within the scope of the present invention. For example, collecting portion 3 may be divided with more than one corresponding aperture so that different types materials such as sharps, excised tissue, swabs etc can be collected in different portions of the collecting portion.

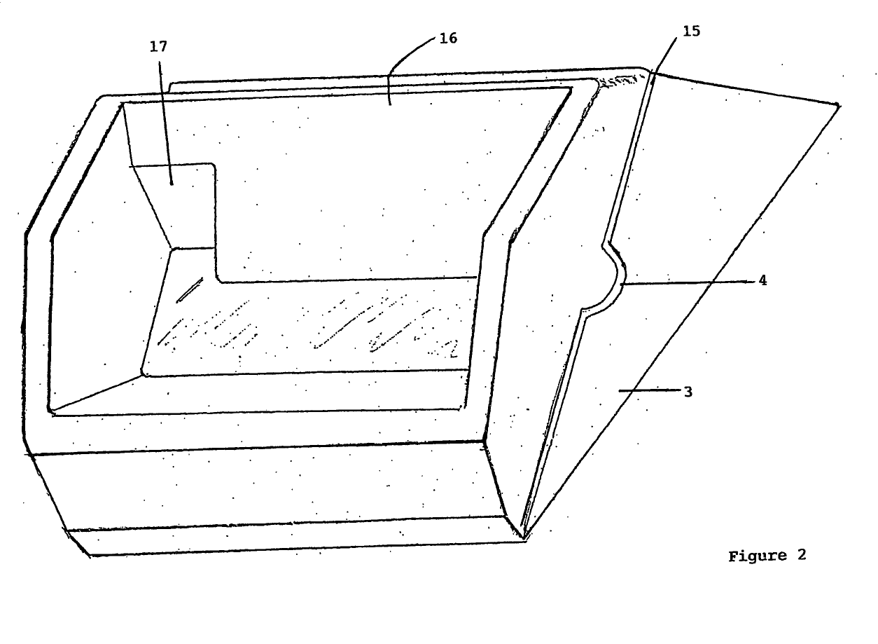

FIG. 2 depicts collecting portion 3 of FIG. 1 in perspective view. Indentation 4 as previously described is depicted. Furthermore edge 15 provides a surface for holding portion 2 to sit on when holding portion 2 is placed over collecting portion 3. Collecting portion 3 defines a cavity 16 which serves as a vessel to hold surgical waste such as used swabs, excised tissue and body fluids. Recess 17 in the inside of collecting portion 3 enables a vessel such as a disposable cup to be placed in aperture 14 of holding portion 2. Such a cup may be used for a variety of purposes. It may be used to contain waste fluids or waste material during surgery. It may also may be filled with sterile saline solution which may be used to irrigate wounds or surgical sites during surgery. It is apparent that the collecting portion according to this embodiment is detachable to enable it to be separately disposed of, or alternatively emptied using aseptic technique and returned to the surgical tray.

The collecting portion may also take another form, for example, it may comprise a tray which may or may not be slidably engaged with the holding portion in a similar manner to a drawer in a chest of drawers. In addition, the collecting portion according to the present invention may further comprise a fluid absorbent surface, such as a fluid absorbent mat.

FIG. 3 depicts holding portion 2 in perspective view from underneath. Retainers 5 in the form of cavities are clearly visible. It can be seen that they are generally elongate tapered in shape with an elongate bi-lobed cross section. Each of cavities or retainers 5 ends in an aperture 18. Broken lines 8 from FIG. 1 are represented by line 19 which depicts the cross sectional shape of the cavity or retainers. Apertures 6 and 14 and indentation 13 are also visible from the underside of holding portion 2.

Retainers 5 can be adapted to retain a variety of different instruments. For example, a person skilled in the art will appreciate that varying the shape of the retainers will enable a variety of different instruments (or sets of instruments) to be retained.

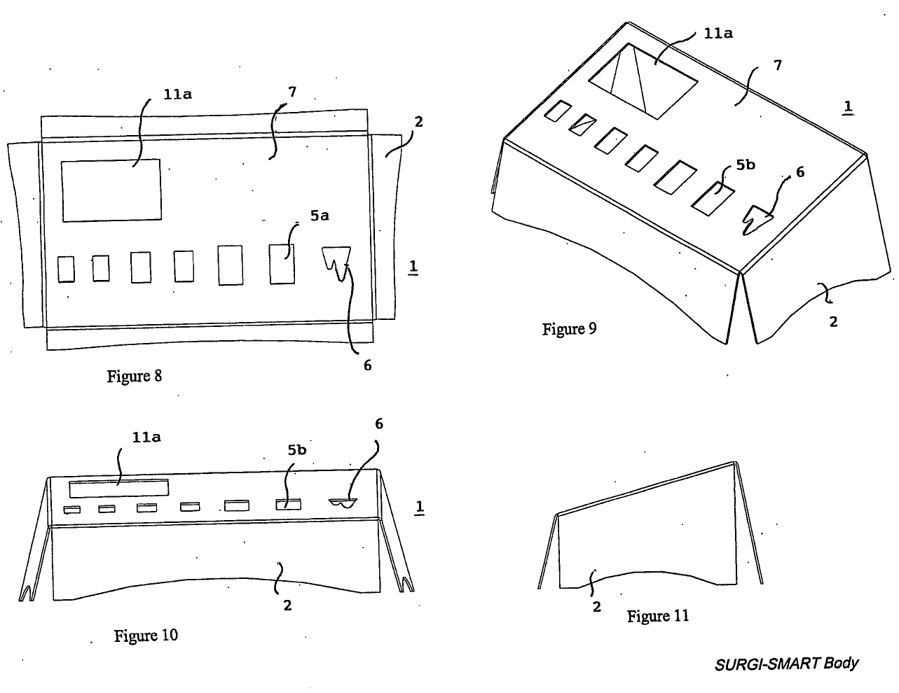

The embodiments depicted in FIGS. 4 to 11 demonstrate a further feature of the present invention. It will be apparent from these figures that the sides of the holder are not connected at the corners. This is because the holder is cut from a single flat sheet of material (such as stainless steel, a polymer or any suitable material), and the sides are bent down to form the stand portion 2a of the holder. The sheet material may be any suitable material as previously described herein. In one preferred embodiment, 4 mm thick plastic is used.

The word ‘comprising’ and forms of the word ‘comprising’ as used in this description do not limit the invention claimed to exclude any variants or additions.

Modifications and improvements to the invention will be readily apparent to those skilled in the art. Such modifications and improvements are intended to be within the scope of this invention.

1. A holder for surgical instruments, said instruments being of the type having a distal portion for engagement with a patient during surgery and a proximal portion for manipulation by a surgeon, said holder comprising a body, a plurality of retainers mounted on or in the body and a separator located between adjacent retainers, wherein location of the distal portion of a surgical instrument in or by one of the retainers substantially separates the distal portion of the surgical instrument against contact with the distal portion of a surgical instrument located in an adjacent other of the retainers and the distal portion of the surgical instrument being substantially enclosed by the retainer.

2. (canceled)

3. A holder according to claim 1 wherein the retainer defines a cavity in the body of the holder.

4. A holder according to claim 1 wherein the retainer is separable from the body of the holder.

5. A holder according to claim 4 further comprising an engagement member to engage the retainer with the holder and a fastener to fasten the retainer to the holder.

6. A holder according to claim 4 wherein the retainer comprises a body which passes through an aperture in the holder and a fastening portion which is fastened to the body of the holder.

7. A holder according to claim 1 wherein the retainer comprises an aperture at its distal end.

8. A holder according to claim 1 wherein the retainer defines an elongated and tapered cavity.

9. A holder according to claim 8 wherein the retainer has an oval or bi-lobed cross section.

10. A holder according to claim 1 comprising a holding portion and a collecting portion.

11. A holder according to claim 10 wherein the collecting portion is detachable.

12. A holder according to claim 10 wherein instruments are held in the retainers so as to not contact the collecting portion.

13. A holder according to claim 10 wherein the holder is shaped so that the holder or the holding portion of the holder is stackable with a plurality of like-shaped holders or holding portions.

14. A holder according to claim 10 wherein the collecting portion is shaped so that it is stackable with a plurality of like-shaped collecting portions.

15. A holder according to claim 1 comprising an engagement portion to engage another surgical tray or holder.

16. A holder according to claim 1 further comprising a portion for holding other surgical items.

17. A holder according to claim 1 further comprising a device for removal of medical sharps from medical implements comprising a body defining an aperture wherein at least one portion of the body projects into the aperture and said portion comprises a lip defining the aperture so as to engage the medical sharp for removal from the medical implement.

18. A holder according to claim 17 wherein the aperture is a generally rounded rectangular shape and is missing one corner due to the projection of the body into the aperture.

19. A holder according to claim 17 wherein the lip extends:
(a) less than half way around the perimeter of the aperture; and
(b) half way across the projection from the body into the aperture.

20. A holder according to claim 17 wherein a portion of the body comprises a tapered or serrated edge to enable removal of a screw-threaded sharps from medical instruments.

21. A device for removal of medical sharps from medical implements comprising a body defining an aperture wherein at least one portion of the body projects into the aperture and said portion comprises a lip extending substantially perpendicular to the plane of the body defining the aperture so as to engage the medical sharp for removal from the medical implement.

22. A device according to claim 21 wherein the aperture is a generally rounded rectangular shape and is missing one corner due to the projection of the body into the aperture.

23. A device according to claim 21 wherein the lip extends:
(a) less than half way around the perimeter of the aperture; and
(b) half way across the projection from the body into the aperture.

24. A device according to claim 23 wherein a portion of the body comprises a tapered or serrated edge to enable removal of screw-threaded sharps from medical instruments.

25. A retainer suitable for use with a holder for surgical instruments according to claim 1 wherein the distal portion of a surgical instruments retained in the retainer is enclosed by the retainer.

26. A retainer according to claim 25 wherein the retainer defines a cavity in the body of the holder.

27. A retainer according to claim 25 capable of being separately reversibly fastened in apertures in the body of a holder.

28. A retainer according to claim 25 comprising an aperture at the distal end.

29. A retainer according to claim 25 comprising an elongated and tapered cavity.

30. A retainer according to claim 29 wherein the cavity has an oval or bi-lobed cross section.

31. A retainer according to claim 25 which retains an instrument by engaging it.

32-37. (canceled)
